(19) United States
(12) Patent Application Publication
(10) Pub. No.: US 2011/0046730 A1
(43) Pub. Date: Feb. 24, 2011

(54) IMPLANTABLE MICROPHONE SYSTEM

(76) Inventor: Werner Meskens, Opwijk (BE)

Correspondence Address: JOHN S. PRATT, ESQ. (Cochlear);KILPATRICK STOCKTON LLP, 1100 PEACHTREE STREET, SUITE 2800, ATLANTA, GA, 30309, US

(21) Appl. No.: 12/935,903

(22) PCT Filed: Mar. 31, 2009
(86) PCT No.: PCT/US09/38947

§ 371 Date: Sep. 30, 2010

(30) Foreign Application Priority Data
Mar. 31, 2008 (AU) 2008901547

(51) Int. Cl. A61F 2/18; A61N 1/00

(52) U.S. Cl. 623/10; 607/55

(57) ABSTRACT

An at least partially implantable hearing prosthesis. The hearing prosthesis comprises an implantable internal energy transfer assembly configured to receive power from an external device and having an implantable microphone system removably positioned therein configured to receive a sound signal and to generate electrical signals representing the received sound signal; a main implantable component having a sound processing unit configured to convert the electrical signals into data signals; and an output stimulator configured to stimulate the recipient's ear based on the data signals.

### CROSS-REFERENCE TO RELATED APPLICATIONS

The present application is a National Stage Application of International Application No. PCT/US09/038,947, filed Mar. 31, 2009, and claims the benefit of Australian Provisional Application No. 2008901547; filed Mar. 31, 2008. The contents of these applications are hereby incorporated by reference herein.

### BACKGROUND

1. Field of the Invention

The present invention relates generally to an implantable hearing prosthesis and, more particularly, to an implantable microphone system.

2. Related Art

Medical devices having one or more implantable components, generally referred to as implantable medical devices, have provided a wide range of therapeutic benefits to patients over recent decades. Implantable hearing prostheses that treat the hearing loss of a prosthesis recipient are one particular type of implantable medical devices that are widely used today.

Hearing loss, which may be due to many different causes, is generally of two types, conductive and sensorineural. In some cases, a person suffers from hearing loss of both types. Conductive hearing loss occurs when the normal mechanical pathways for sound to reach the cochlea, and thus the sensory hair cells therein, are impeded, for example, by damage to the ossicles. Individuals who suffer from conductive hearing loss typically have some form of residual hearing because the hair cells in the cochlea are undamaged. As a result, individuals suffering from conductive hearing loss typically receive an implantable hearing prosthesis that generates mechanical motion of the cochlea fluid. Some such hearing prosthesis, such as acoustic hearing aids, middle ear implants, etc., include one or more components implanted in the recipient, and are referred to herein as implantable hearing prosthesis.

In many people who are profoundly deaf, however, the reason for their deafness is sensorineural hearing loss. Sensorineural hearing loss occurs when there is damage to the inner ear, or to the nerve pathways from the inner ear to the brain. As such, those suffering from some forms of sensorineural hearing loss are thus unable to derive suitable benefit from hearing prostheses that generate mechanical motion of the cochlea fluid. As a result, implantable hearing prostheses that deliver electrical stimulation to nerve cells of the recipient's auditory system have been developed to provide the sensations of hearing to persons whom do not derive adequate benefit from conventional hearing aids. Such electrically-stimulating hearing prostheses deliver electrical stimulation to nerve cells of the recipient's auditory system thereby providing the recipient with a hearing percept.

As used herein, the recipient's auditory system includes all sensory system components used to perceive a sound signal, such as hearing sensation receptors, neural pathways, including the auditory nerve and spiral ganglion, and parts of the brain used to sense sounds. Electrically-stimulating hearing prostheses include, for example, auditory brain stimulators and cochlear prostheses (commonly referred to as cochlear prosthetic devices, cochlear implants, cochlear devices, and the like; simply “cochlear implants” herein.)

Oftentimes sensorineural hearing loss is due to the absence or destruction of the cochlear hair cells which transduce acoustic signals into nerve impulses. It is for this purpose that cochlear implants have been developed. Cochlear implants provide a recipient with a hearing percept by delivering electrical stimulation signals directly to the auditory nerve cells, thereby bypassing absent or defective hair cells that normally transduce acoustic vibrations into neural activity. Such devices generally use an electrode array implanted in the cochlea so that the electrodes may differentially activate auditory neurons that normally encode differential pitches of sound.

Auditory brain stimulators are used to treat a smaller number of recipients with bilateral degeneration of the auditory nerve. For such recipients, the auditory brain stimulator provides stimulation of the cochlear nucleus in the brainstem.

Totally or fully implantable forms of the above and other implantable hearing prostheses have been developed to treat a recipient's conductive, sensorineural and/or combination hearing loss. As used herein, a totally implantable hearing prosthesis refers to an implantable prosthesis that is capable of operating, at least for a period of time, without the need for any external device.

### SUMMARY

In one aspect of the present invention, a cochlear implant totally implantable in a recipient is provided. The cochlear implant comprises: an internal energy transfer assembly configured to receive power from an external device and having an implantable microphone system removably positioned therein configured to receive a sound signal and to generate electrical signals representing the received sound signal; a main implantable component having a sound processing unit configured to convert the electrical signals into data signals; and an electrode assembly implantable in the recipient's cochlea configured to deliver to the cochlea electrical stimulation signals generated based on the data signals.

In another aspect of the present invention, a hearing prosthesis at least partially implantable in a recipient is provided. The hearing prosthesis comprises an implantable internal energy transfer assembly configured to receive power from an external device and having an implantable microphone system removably positioned therein configured to receive a sound signal and to generate electrical signals representing the received sound signal; a main implantable component having a sound processing unit configured to convert the electrical signals into data signals; and an output stimulator configured to stimulate the recipient's ear based on the data signals.

In a still other aspect of the present invention, a method for evoking a hearing percept in a recipient is provided. The method comprises: receiving a sound signal via an implantable microphone system removably positioned in an implantable internal energy transfer assembly configured to receive power from an external device; providing an electrical signal representing the sound signal to a main implantable component having a sound processing unit; converting, with the sound processing unit, the electrical signal representing the sound signal into one or more data signals; stimulating the recipient's ear based on the one or more data signals.

### BRIEF DESCRIPTION OF THE DRAWINGS

Embodiments of the present invention are described below with reference to the attached drawings, in which:

FIG. 1 is a perspective view of an exemplary totally implantable cochlear implant, in which embodiments of the present invention may be implemented;

FIG. 2 is a functional block diagram of a totally implant cochlear implant in accordance with embodiments of the present invention shown with an external device;

FIG. 3A is a schematic diagram of the cochlear implant of FIG. 2 in accordance with embodiments of the present invention;

FIG. 3B is a schematic diagram of the cochlear implant of FIG. 2 in accordance with embodiments of the present invention;

FIG. 4 is a perspective view of a totally implant cochlear implant in accordance with embodiments of FIG. 2;

FIG. 5A is a cross-sectional view of an internal energy transfer assembly in accordance with embodiments of FIG. 4;

FIG. 5B is a cross-sectional, enlarged view of an implantable microphone system in accordance with embodiments of FIG. 5A;

FIG. 6A is a cross-sectional view of an internal energy transfer assembly in accordance with embodiments of FIG. 4;

FIG. 6B is a cross-sectional, enlarged view of an implantable microphone system in accordance with embodiments of FIG. 6A;

FIG. 7 is schematic diagram of a cochlear implant in accordance with embodiments of the present invention;

FIG. 8A is a diagram illustrating successive times time slots of a subcutaneous transfer link in accordance with embodiments of the present invention;

FIG. 8B is a schematic view of one embodiment of a implantable microphone system illustrating an alternative arrangement for subcutaneous transfer in accordance with embodiments of the present invention; and

FIG. 9 is a flowchart illustrating the operations performed by an implantable hearing prosthesis in accordance with embodiments of the present invention.

### DETAILED DESCRIPTION

Aspects of the present invention are generally directed to an implantable hearing prosthesis in which an implantable microphone is disposed in a structure configured to receive power and/or data from an external device. Specifically, the implantable hearing prosthesis comprises an internal energy transfer assembly to receive the power and/or data from the external device. Disposed in the internal energy transfer assembly is an implantable microphone system which functions as the hearing prosthesis sound pickup component. The implantable microphone system includes a microphone disposed in a magnet that is used to align the internal energy transfer assembly and the external device during power and/or data transfer.

Embodiments of the present invention are described herein primarily in connection with one type of implantable hearing prosthesis, namely a totally or fully implantable cochlear prosthesis (commonly referred to as a cochlear prosthetic device, cochlear implant, cochlear device, and the like; simply “cochlear implants” herein). As used herein, a totally implantable cochlear implant refers to an implant that is capable of operating, at least for a period of time, without the need for any external device. It would be appreciated that embodiments of the present invention may also be implemented in a cochlear implant that includes one or more external components. It would be further appreciated that embodiments of the present invention may be implemented in any partially or fully implantable hearing prosthesis now known or later developed, including, but not limited to, acoustic hearing aids, auditory brain stimulators, middle ear mechanical stimulators, hybrid electro-acoustic prosthesis or other prosthesis that electrically, acoustically and/or mechanically stimulate components of the recipient's outer, middle or inner ear or in which it may be useful to align an external device with an implanted component.

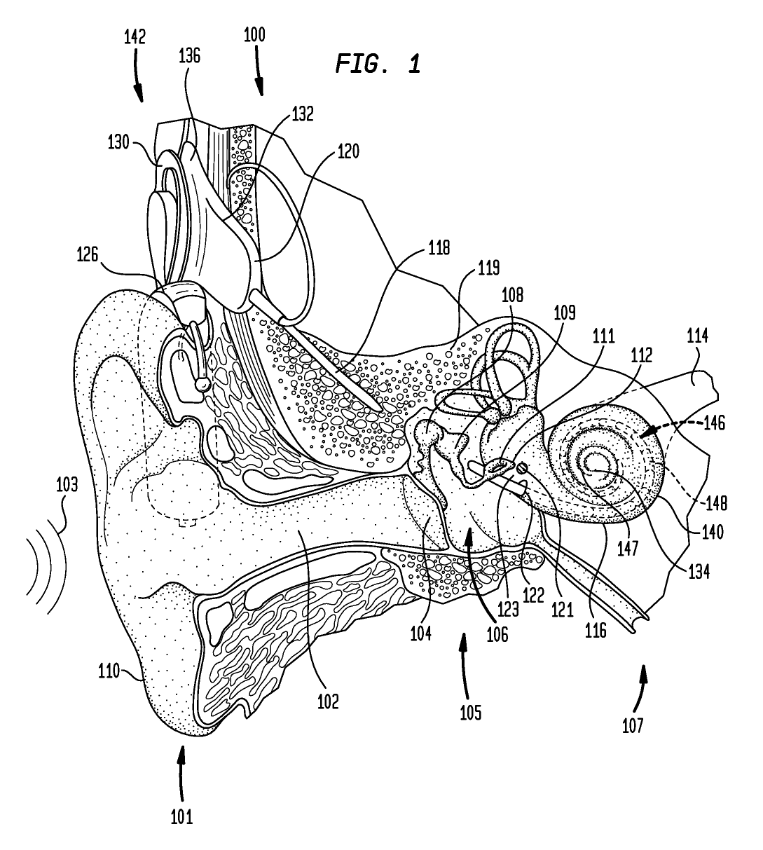

FIG. 1 is perspective view of a totally implantable cochlear implant, referred to as cochlear implant 100, implanted in a recipient. The recipient has an outer ear 101, a middle ear 105 and an inner ear 107. Components of outer ear 101, middle ear 105 and inner ear 107 are described below, followed by a description of cochlear implant 100.

In a fully functional ear, outer ear 101 comprises an auricle 110 and an ear canal 102. An acoustic pressure or sound wave 103 is collected by auricle 110 and channeled into and through ear canal 102. Disposed across the distal end of ear cannel 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109 and the stapes 111. Bones 108, 109 and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound.

As shown, cochlear implant 100 comprises one or more components which are temporarily or permanently implanted in the recipient. Cochlear implant 100 is shown in FIG. 1 with an external device 142 which, as described below, is configured to provide power to the cochlear implant.

In the illustrative arrangement of FIG. 1, external device 142 may comprise a power source (not shown) disposed in a Behind-The-Ear (BTE) unit 126. External device 142 also includes components of a transcutaneous energy transfer link, referred to as an external energy transfer assembly. The transcutaneous energy transfer link is used to transfer power and/or data to cochlear implant 100. As would be appreciated, various types of energy transfer, such as infrared (IR), electromagnetic, capacitive and inductive transfer, may be used to transfer the power and/or data from external device 142 to cochlear implant 100. In the illustrative embodiments of FIG. 1, the external energy transfer assembly comprises an external coil 130 that forms part of an inductive radio frequency (RF) communication link. External coil 130 is typically a wire antenna coil comprised of multiple turns of electrically insulated single-strand or multi-strand platinum or gold wire. External device 142 also includes a magnet (not shown) positioned within the turns of wire of external coil 130. It should be appreciated that the external device shown in FIG. 1 is merely illustrative, and other external devices may be used with embodiments of the present invention.

Cochlear implant 100 comprises an internal energy transfer assembly 132 which may be positioned in a recess of the temporal bone adjacent auricle 110 of the recipient. As detailed below, internal energy transfer assembly 132 is a component of the transcutaneous energy transfer link and receives power and/or data from external device 142. In the illustrative embodiment, the energy transfer link comprises an inductive RF link, and internal energy transfer assembly 132 comprises a primary internal coil 136. Internal coil 136 is typically a wire antenna coil comprised of multiple turns of electrically insulated single-strand or multi-strand platinum or gold wire. Positioned substantially within the wire coils is an implantable microphone system (not shown). As described in detail below, the implantable microphone assembly includes a microphone (not shown), and a magnet (also not shown) fixed relative to the internal coil.

Cochlear implant 100 further comprises a main implantable component 120 and an elongate electrode assembly 118. In embodiments of the present invention, internal energy transfer assembly 132 and main implantable component 120 are hermetically sealed within a biocompatible housing. In embodiments of the present invention, main implantable component 120 includes a sound processing unit (not shown) to convert the sound signals received by the implantable microphone in internal energy transfer assembly 132 to data signals. Main implantable component 120 further includes a stimulator unit (also not shown) which generates electrical stimulation signals based on the data signals. The electrical stimulation signals are delivered to the recipient via elongate electrode assembly 118.

Elongate electrode assembly 118 has a proximal end connected to main implantable component 120, and a distal end implanted in cochlea 140. Electrode assembly 118 extends from main implantable component 120 to cochlea 140 through mastoid bone 119. In some embodiments electrode assembly 118 may be implanted at least in basal region 116, and sometimes further. For example, electrode assembly 118 may extend towards apical end of cochlea 140, referred to as cochlea apex 134. In certain circumstances, electrode assembly 118 may be inserted into cochlea 140 via a cochleostomy 122. In other circumstances, a cochleostomy may be formed through round window 121, oval window 112, the promontory 123 or through an apical turn 147 of cochlea 140.

Electrode assembly 118 comprises a longitudinally aligned and distally extending array 146 of electrodes 148, sometimes referred to as electrode array 146 herein, disposed along a length thereof. Although electrode array 146 may be disposed on electrode assembly 118, in most practical applications, electrode array 146 is integrated into electrode assembly 118. As such, electrode array 146 is referred to herein as being disposed in electrode assembly 118. As noted, a stimulator unit generates stimulation signals which are applied by electrodes 148 to cochlea 140, thereby stimulating auditory nerve 114.

As noted, cochlear implant 100 comprises a totally implantable prosthesis that is capable of operating, at least for a period of time, without the need for external device 142. Therefore, cochlear implant 100 further comprises a rechargeable power source (not shown) that stores power received from external device 142. The power source may comprise, for example, a rechargeable battery. During operation of cochlear implant 100, the power stored by the power source is distributed to the various other implanted components as needed. The power source may be located in main implantable component 120, or disposed in a separate implanted location.

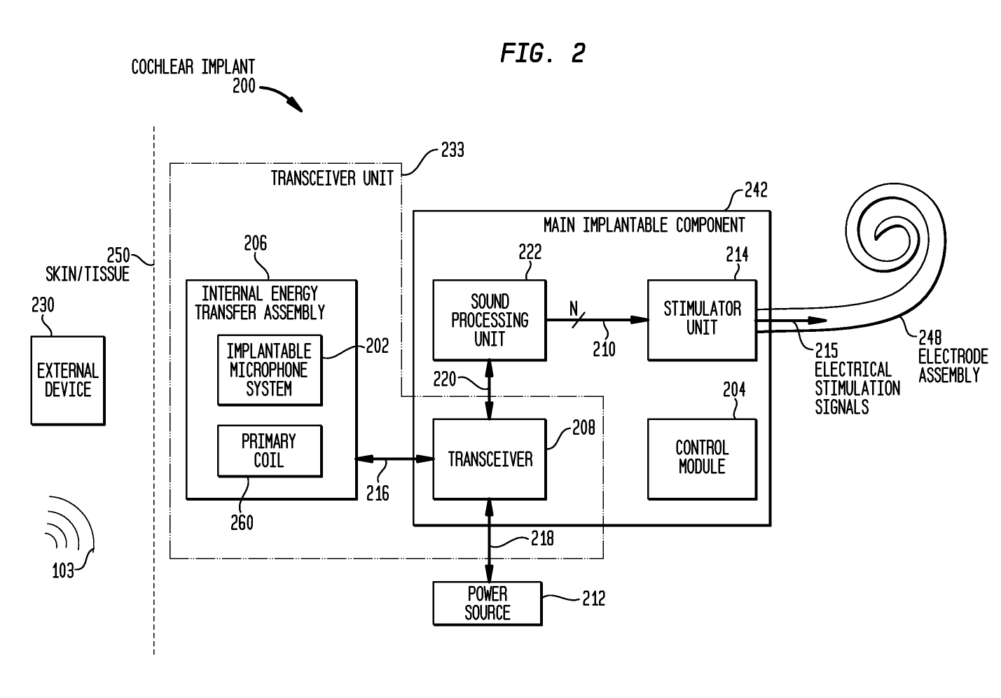

FIG. 2 is a functional block diagram of embodiments of cochlear implant 100 in which embodiments of the present invention may be implemented, referred to as cochlear implant 200 herein. Similar to the above embodiments, cochlear implant 200 is totally implantable; that is, all components of cochlear implant 200 are configured to be implanted under skin/tissue 250 of a recipient. Because all components of cochlear implant 200 are implantable, cochlear implant 200 operates, for at least a period of time, without the need of an external device, such as external device 230.

Cochlear implant 200 comprises a transceiver unit 233, a main implantable component 242, a rechargeable power source 212, and an electrode assembly 248. The embodiments of FIG. 2 are illustrative, and it would be appreciated that one or more components may be disposed in the same or different housings.

As shown in FIG. 2, transceiver unit 233 comprises an internal energy transfer assembly 206 and a transceiver 208. As discussed below, internal energy transfer assembly 206 and transceiver 208 cooperate to receive power and/or data from external device 230. As used herein, transceiver unit 233 refers to any collection of one or more implanted components which form part of a transcutaneous energy transfer system. Furthermore, internal energy transfer assembly 206 refers to an assembly which includes the component(s) of a transceiver unit 233 which transmit and receive data, such as, for example a coil for a magnetic inductive arrangement, an antenna for an alternative RF system, capacitive plates, or any other suitable arrangement. As such, in embodiments of the present invention, various types of energy transfer, such as infrared (IR), electromagnetic, capacitive and inductive transfer, may be used to transfer the power and/or data from external device 230 to cochlear implant 200.

In the illustrative embodiments of FIG. 2, inductive transfer is used to transfer power and/or data, and internal energy transfer assembly 206 comprises a primary coil 260. Transceiver 208 is connected to primary coil 260 and comprises the circuit elements used to decode and distribute the received power and/or data. The electrical connection between primary coil 260 and transceiver 208 is illustrated by bidirectional arrow 216. In the embodiments of FIG. 2, transceiver 208 is physically disposed in main implantable component 242.

As shown, internal energy transfer assembly 206 comprises an implantable microphone system 202. As described in detail below, implantable microphone system 202 comprises a magnet (not shown), a microphone configured to sense a sound signal 103, and one or more components to pre-process the microphone output. An electrical signal representing the pre-processed output of the microphone, referred to as pre-processed microphone output or signals herein, is provided from transceiver unit 233 to a sound processing unit 222 in main implantable component 242. For ease of illustration, the transfer of the pre-processed sound signal is not shown in FIG. 2, but is described in greater detail below.

Cochlear implant 200 further comprises main implantable component 242. As noted, main implantable component 242 includes transceiver 208 and sound processing unit 222. Main implantable component 242 further includes stimulator unit 214 and control module 204. As noted, the pre-processed microphone output is provided to sound processing unit 222. Sound processing unit 22 implements one or more speech processing and/or coding strategies to convert the pre-processed microphone output into data signals 210 which are provided to a stimulator unit 214. Based on data signals 210, stimulator unit 214 generates electrical stimulation signals 215 for delivery to the cochlea of the recipient. In the embodiment illustrated in FIG. 2, cochlear implant 200 comprises an embodiment of electrode assembly 118 of FIG. 1, referred to as electrode assembly 248, for delivering stimulation signal 215 to the cochlea.

Cochlear implant 200 also includes rechargeable power source 212. Power source 212 may comprise, for example, one or more rechargeable batteries. As noted above, power is received from external device 230, and is distributed immediately to desired components, or is stored in power source 212. The power may then be distributed to the other components of cochlear implant 200 as needed for operation.

As noted, main implantable component 242 further comprises control module 204. Control 204 includes various components for controlling the operation of cochlear implant 200, or for controlling specific components of cochlear implant 200. For example, controller 204 may control the delivery of power from power source 212 to other components of cochlear implant 200.

For ease of illustration, internal energy transfer assembly 206, main implantable component 242 and power source 212 are shown separate. It would be appreciated that one or more of the illustrated elements may be integrated into a single housing or share operational components. For example, in certain embodiments of the present invention, internal energy transfer assembly 206, main implantable component 242 and power source 212 may be integrated into a hermetically sealed housing.

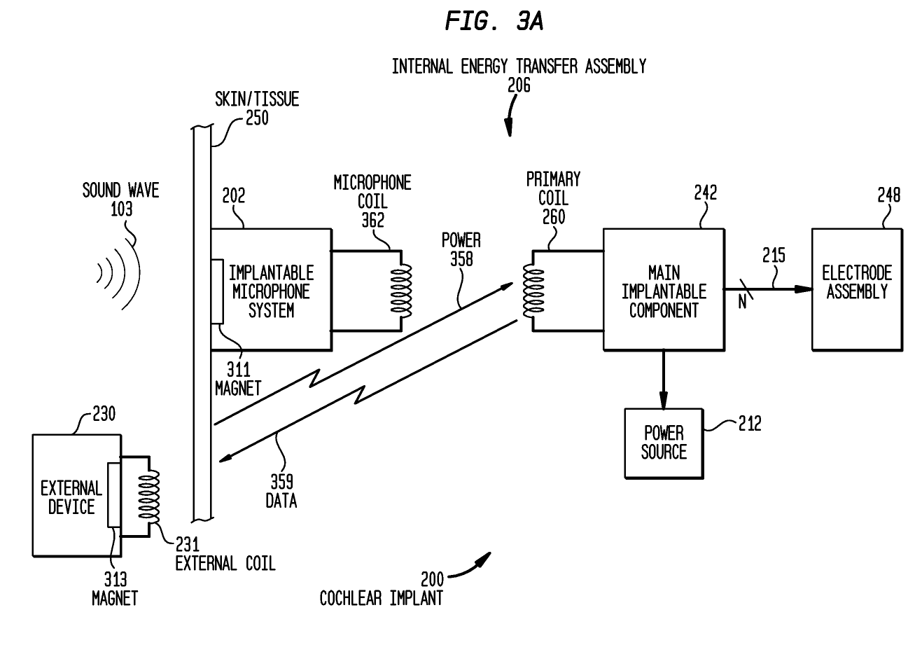

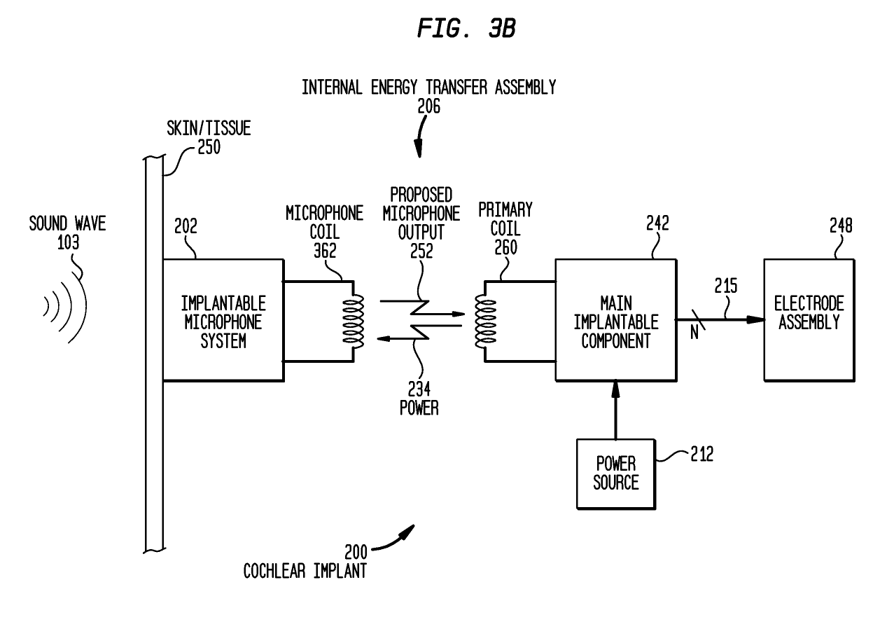

FIG. 3A is a schematic diagram of cochlear implant 200 in accordance with embodiments of the present invention. As previously noted, cochlear implant 200 comprises internal energy transfer assembly 206, main implantable component 242, power source 212 and electrode assembly 248. For ease of illustration, electrode assembly 248 is shown as a block 248.

In the illustrative embodiments of the present invention, an inductive transcutaneous communication link is used to transfer power and/or data between external device 230 and cochlear implant 200. As discussed in greater detail below, in embodiments of the present invention, the inductive communication link comprises a bi-directional communication link. That is, power 358 and/or data are transferred from external device 230 to cochlear implant 200, while cochlear implant 200 is configured to transfer data 359 to the external device. Internal energy transfer assembly 206 comprises primary coil 260 which receives the power/data, and which transmits data to external device 230. The inductive communication link between external device 230 and cochlear implant 200 is provided between external coil 231 and primary coil 260.

As noted, primary coil 260 is a wire antenna coil comprised of multiple turns of electrically insulated single-strand or multi-strand platinum or gold wire. Implantable microphone system 202 is positioned substantially within the wire coil(s). In other words, the coil(s) of wire extends around a portion of implantable microphone assembly 202. However, to facilitate understanding of embodiments of the present invention, implantable microphone assembly 202 is shown separated from primary coil 260.

In conventional cochlear implants, a microphone is positioned externally to the recipient to sense a sound 103. In such conventional systems, the sound is processed and transmitted as an electrical signal to the implanted components. However, the requirement of an external microphone has practical and aesthetic disadvantages for a recipient. Embodiments of the present invention overcome these disadvantages through the use of implantable microphone assembly 202. As shown, implantable microphone assembly 202 is implanted underneath and adjacent to the recipient's skin and/or tissue 250.

As is well known, sound travels as a propagating wave through air or other medium. In FIG. 3A, sound wave 103 impinges upon skin/tissue 250, and generates a corresponding sound wave within the recipient, specifically in skin/tissue 250, that is detected by an acoustic transducer (not shown), generally referred to as a microphone herein, within implantable microphone assembly 202. The microphone converts the sound wave propagating through the recipient into an electrical output. In embodiments of the present invention, the microphone is disposed adjacent or in contact with skin/tissue 250 in order to minimize attenuation and provide the best signal to noise ratio.

As detailed below, in embodiments of the present invention, implantable microphone system 202 is configured to pre-process the electrical signal output by the microphone, and provide a representation of the pre-processed microphone signal, referred to herein as pre-processed microphone output or signals, to main implantable component 242 for processing and conversion into electrical stimulation signals 215. There are several ways in which the pre-processed microphone output may be provided to main implantable component 242. For example, in certain embodiments, a direct electrical connection may be provided between implantable microphone system 202 and main implantable component 242. In such embodiments, implantable microphone system 202 is connected to main implantable component 242 by one or more wires.

In alternative embodiments, pre-processed microphone signals are provided to main implantable component 242 using a subcutaneous wireless energy transfer link. Various types of wireless transfer links, including an infrared (IR) link, electromagnetic link, capacitive link, inductive link, etc., may be used. FIG. 3B illustrates specific embodiments of the present invention in which an inductive energy transfer link is used to transfer the pre-processed microphone signals to main implantable component 242. As shown, implantable microphone system 202 comprises a microphone coil 362. Microphone coil 362 is a wire antenna coil comprised of multiple turns of electrically insulated single-strand or multi-strand platinum or gold wire. In operation, pre-processed microphone signals, shown as signals 352 in FIG. 3B, are inductively transmitted from microphone coil 362 to primary coil 260 electrically connected to main implantable component 242. In such embodiments in which a wireless link is used to transfer pre-processed microphone signals 352, implantable microphone system 202, although disposed within coil 260, may remain physically and electrically detached from the other components of cochlear implant 200. That is, although implantable microphone system 202 may be in contact with other components, the microphone system is not physically or electrically connected to the other components. Thus, implantable microphone system 202 is removably positioned in internal energy transfer assembly 206, thereby providing a surgeon or other individual with the ability to easily remove implantable microphone system 202 with affecting the implanted position or operation of any other implanted component.

As noted, in certain embodiments of the present invention, the output of the microphone is pre-processed prior to providing the microphone output to main implantable component 242 for additional processing. Pre-processing of the microphone output may include amplification, filtering, etc., and one or more pre-processing components may be required. As would be appreciated, the pre-processing and transferring of the microphone output requires at least some power to be provided. As described below, this power is provided by a local rechargeable power source (not shown) within implantable microphone system 202. Similar to the transfer of pre-processed microphone signals 352 discussed above, the rechargeable power source within implantable microphone system 202 may be recharged using several methods, including a direct electrical connection with main implantable component 242, or through a wireless link with the main implantable component. FIG. 3B illustrates embodiments in which a wireless link is used to recharge the local power source within implantable microphone system 202. Specifically, in the embodiments of FIG. 3B, power 234 is provided from primary coil 260 to microphone coil 362. The transferred power is then used to recharge the local power source.

In cochlear implants using the transcutaneous transfer of power, it is generally desirable to position the external power transmitting device, external coil 231 in FIG. 3A, in close proximity to the internal receiving device, primary coil 260 in FIG. 3A. Proximity of the transmitting and receiving devices is generally preferable to provide efficient power transfer. However, it may be difficult for a recipient or other user to accurately determine the position of the implanted receiving element, thus making it difficult to correctly position the external transmitting device for efficient transfer. To solve this problem, magnets are generally provided in or near the internal receiving device and the external power transmitting device. The magnets provide an attractive force that aligns the devices, thereby enabling the efficient transfer of power.

FIG. 3A is an illustrative embodiment of transmitting and receiving devices which use magnets to align the devices. As shown, external device 230 includes a magnet 313 and implantable microphone system 202 includes a magnet 311. In operation, magnets 311 and 313 would ensure that external coil 231 is positioned proximate to primary coil 260 in order to provide efficient power transfer. In particular, magnets 311 and 313 ensure that primary coil 260 and external coil 231 are positioned substantially parallel on opposite sides of skin/tissue 250. For ease of illustration, external coil 231 and primary coil 260 are not shown in FIG. 3A in the typical positions for power transfer. Thus, it should be appreciated that the transfer of power 359 in FIG. 3A is merely illustrative.

As would be appreciated, external device 230 may comprise a variety of devices which have the ability to transmit power to cochlear implant 200. For example, as described above with reference to FIG. 1, external device 230 may comprises a housing that is configured to be worn behind the ear of the recipient, referred to as behind-the-ear (BTE) unit. The BTE unit has therein a power source, a power transmitter. Connected to the power transmitter via a cable is coil 231. In alternative embodiments, external device comprises a housing having a power source, a power transmitter and a magnet positioned therein. Attached to the exterior of the housing is coil 231. The housing is configured to be secured to the recipient via a magnetic coupling with magnet 311 in cochlear implant 200.

It should also be appreciated that in certain embodiments external device 230 may be used for purposes other than providing power to cochlear implant 200. For example, in one such embodiment, external device 230 includes an external sound input element that may be used to provide a sound signal to cochlear implant 200. It would be appreciated that a sound input element in accordance with embodiments of the present invention may comprise a microphone, an electrical input which connects cochlear implant 200 for example, FM hearing systems, MP3 players, televisions, mobile phones, etc. Furthermore, in other such embodiments, external device 230 may, as noted, receive operational telemetry or other performance data from cochlear implant 200, or the external device may transfer data, such as software revisions, altered operational data, commands from a user remote control or health professional programming device, etc., to cochlear implant 200.

In the embodiments of FIG. 3B, cochlear implant 200 uses microphone coil 362 and primary coil 260 to provide the bi-directional transfer of power 234 and pre-processed microphone signals 352. As discussed below, there are several subcutaneous transfer schemes in which bi-directional transfer of power and data may be performed using the same transmitting/receiving elements as is shown in FIG. 3B.

As noted above, the embodiments of cochlear implant 200 shown in FIG. 3A utilize primary coil 260 to transmit data 359 to, and receive power 358 from external device 230. In the embodiments of FIG. 3B, cochlear implant 200 utilizes primary coil to transmit power 234 to, and receive pre-processed microphone signals 352 from implantable microphone system 202. The embodiments of FIGS. 3A and 3B may be conceptually considered first and second modes of operation of cochlear implant 200. In the first mode of operation shown in FIG. 3A, sometimes referred to herein as a recharge mode of operation, cochlear implant 200 is primarily receiving power from external device 230 to recharge power source 212. In the recharge mode of operation, implantable microphone system 202 does not transmit microphone signals to main implantable component 242. Cochlear implant 200 may enter the recharge mode of operation when, for example, external device 230 is brought into proximity to cochlear implant 200. In other embodiments, cochlear implant 200 may enter the recharge mode of operation when the recipient or other user provides the cochlear implant with an input indicating a recharge of power source is desired. In still other embodiments, cochlear implant 200 may automatically enter the recharge mode of operation when the power source reaches a predetermined level, or when a sound signal has not been received by the implant for a predetermined period of time. Cochlear implant 200 may be further configured to enter the recharge mode of operation only when the implant is capable of receiving power from external device 230.

Conversely, in the second mode of operation shown in FIG. 3B, sometimes referred to as a sound delivery mode of operation, cochlear implant 200 is primarily transferring pr-processed microphone signals and power between implantable microphone system 202 and main implantable component 242. In the sound delivery mode of operation, main implantable component 242 does not receive power from external device 230. Cochlear implant 200 may enter or remain in the sound delivery mode of operation when, for example, external device 230 is not in proximity to cochlear implant 200. In other embodiments, cochlear implant 200 may enter the sound delivery mode of operation based on a user input. In still other embodiments, cochlear implant 200 may automatically enter the sound delivery mode of operation when a sound signal is received by the implant.

As noted, the specific embodiments of FIGS. 3A and 3B cochlear implant 200 use two modes of operation to alternatively implement a transcutaneous link and a subcutaneous communication. It should be appreciated that in alternative embodiments of the present invention cochlear implant 200 is configured to concurrently implement a transcutaneous link and subcutaneous link. In one such embodiment, a first type of wireless of communication is used to implement the transcutaneous link, while a second type of link is used to implement the subcutaneous link. In an alternative such embodiment, an interleaving communication scheme may implemented in which external device 230, main implantable component 242 and implantable microphone system 202 share a single communication link to transfer power and data. An exemplary interleaving scheme is described in commonly owned and co-pending U.S. patent application Ser. No. 12/391,029, the contents of which are hereby incorporated by reference herein. In still other such embodiments, multiple transmitting/receiving elements may be provided to enable the concurrent transcutaneous and subcutaneous links. In still other embodiments, a physical electrical connection may be provided to transfer power and data between the implanted components, thereby providing for the exclusive use of primary coil 260 for the transcutaneous communication link.

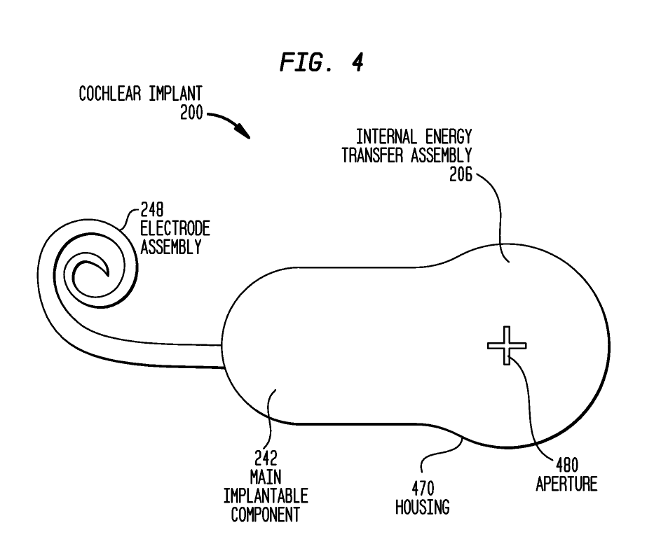

FIG. 4 is a perspective of totally implant cochlear implant 200. As noted above, cochlear implant 200 comprises a biocompatible housing 470 in which internal energy transfer assembly 206 and main implantable component 242 are disposed. Extending from main implantable component 242 is electrode assembly 248. In certain embodiments, housing 470 comprises a hermetically sealed biocompatible housing 470. In embodiments of the present invention, a power source (not shown), such as a rechargeable battery, is provided in main implantable component 242. Alternatively, the power source may be disposed in a separate housing.

As noted above, in embodiments of the present invention, internal energy transfer assembly 206 comprises a coil (not shown) and an implantable microphone system (also not shown). As described in greater detail below, aperture 480 provides an opening in housing 470 in which the implantable microphone system may be positioned.

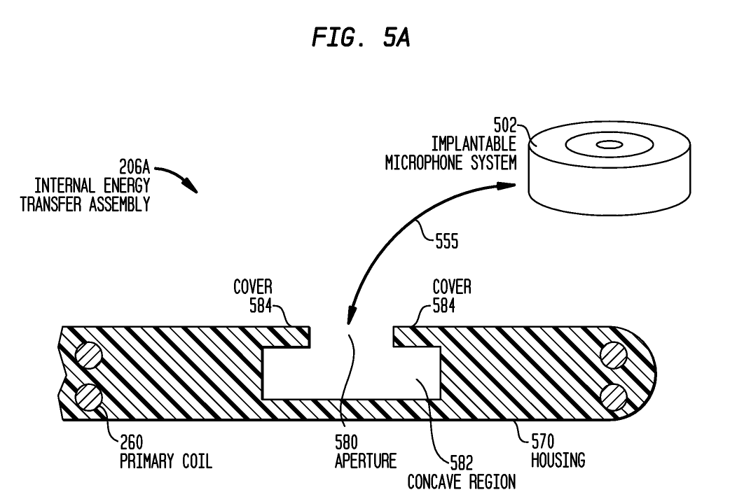

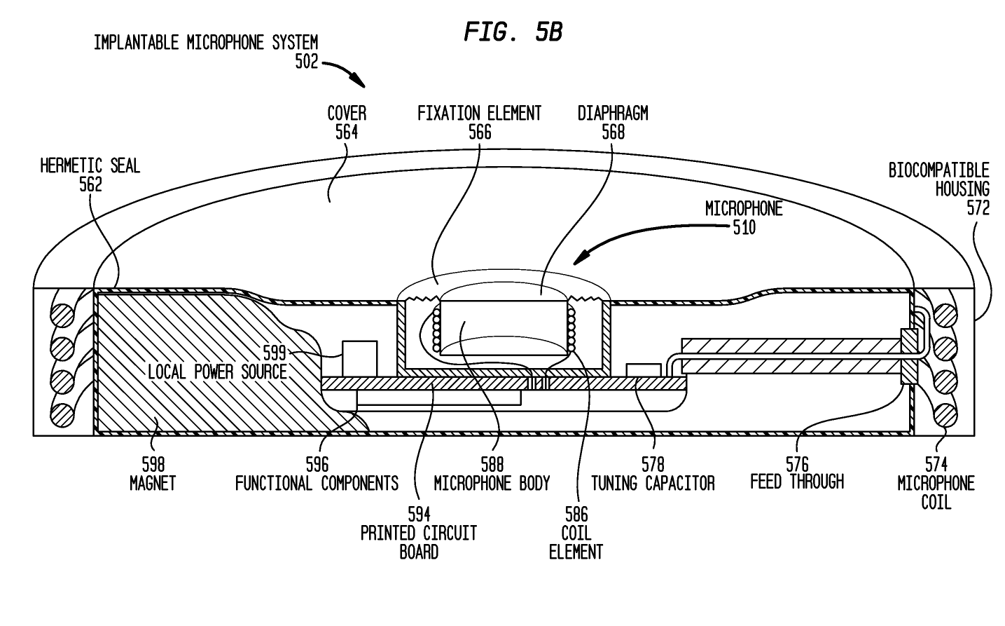

FIG. 5A is a cross-sectional view of internal energy transfer assembly 206, referred to as energy transfer assembly 206A, in accordance with embodiments of the present invention. As noted above, internal energy transfer assembly 206A includes a biocompatible housing 570 in which primary coil 260 is positioned. Wires (not shown) extending from primary coil 260 to components of main implantable component 242 may also be embedded in housing 570. In embodiments, biocompatible housing 570 may comprise any suitable flexible material, such as silicone or other polymer.

As shown in FIG. 5A, housing 570 includes a concave region 582 in which an implantable microphone system 502 may be implanted. Concave region 582 is bound by a portion of biocompatible housing 570, shown as cover 584. Cover 584 has an aperture 580 therein through which implantable microphone system 502 is inserted. As noted housing 570 may comprise a flexible material, thus the orientation of cover 584 may be manipulated to enable insertion of implantable microphone system 502. Once positioned in housing 570, implantable microphone system 502 is substantially surrounded by housing 570, and cover 584 serves to retain in implantable microphone system 502 in the proper position. The positioning of implantable microphone system 502 into concave region 582 is shown by arrow 555. Details of implantable microphone system 502 are provided below with reference to FIG. 5B.

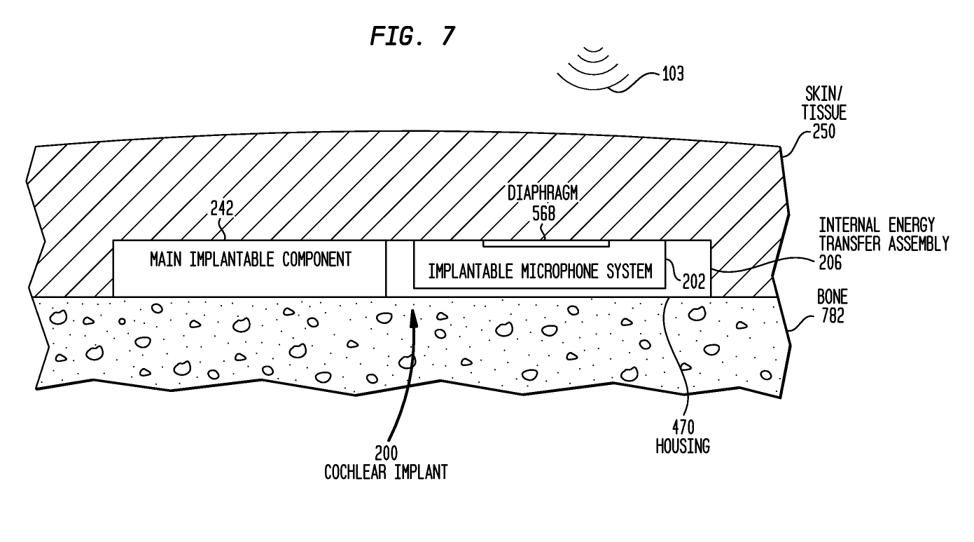

As described with reference to FIG. 5B, in this illustrative embodiment, implantable microphone system 502 is configured to inductively transfer pre-processed microphone signals to main implantable component 242. Because a wireless inductive communication link is utilized, there is no direct, physical electrical connection between implantable microphone system 502 and the remaining components of internal energy transfer assembly 206A. A particular advantage of this implementation is that implantable microphone system 502 may be replaced or upgraded with only minimally invasive surgery. Specifically, as shown with reference to FIG. 7, because implantable microphone system 502 is located near the recipient's skin surface, only a small incision is required to access the system. This provides a surgeon with the ability to easily and safely access implantable microphone system 502, whether for repair, replacement or upgrade.

It would be appreciated that an implanted microphone may be sensitive to both air-conducted sound as well as bone-conducted sound. However, typically in implanted microphones only the air-conducted sound is useful in evaluating a target sound signal, and the body or bone-conducted sounds typically comprises noise that degrades performance of the microphone. For example, body borne sound, such as breathing, may be conducted through the recipient's skull to an implanted microphone. As noted, housing 570 comprises a flexible material. In embodiments of the present invention, housing 570 provides passive vibration isolation for the implantable microphone system 502 and reduce the body conducted sound that is received thereby.

FIG. 5B is a cross-sectional view of implantable microphone system 502 of FIG. 5A. As would be appreciated, the illustrative arrangement of FIG. 5B is not to scale and has been enlarged for ease of illustration.

As shown, implantable microphone system 502 comprises a biocompatible housing 572 which may comprise, for example a flexible material such as silicon or other polymer. As noted, implantable microphone system 502 is configured for inductive communication with main implantable component 242 (FIG. 2). As such, embedded in biocompatible housing is microphone coil 574. As discussed above with reference to FIG. 3B, microphone coil receives power from, and transmits pre-processed microphone signals to, primary coil 260 (FIG. 2).

Implantable microphone system 502 further comprises a magnet 598. As explained above with reference to FIG. 3A, magnet 598 is configured to align an external device with internal energy transfer assembly 206. This alignment enables efficient transfer of power and/or data between the external device and internal energy transfer assembly 206. Disposed in magnet 598 is microphone 510. That is, microphone 510 is at least partially surrounded by magnet 598. For example, in certain embodiments, magnet 598 is positioned around and below microphone 510. In other embodiments, magnet 598 is positioned around microphone 510. In a specific such embodiment, microphone 510 has a circumference which is substantially surrounded by magnet 598. In certain embodiments, at least some spacing between magnet 598 and microphone 510 is provided to minimize electrical losses caused by incidental Eddy currents inside the magnet.

In the illustrative embodiments of FIG. 5B, microphone 510 comprises a magneto-dynamic, or simply dynamic, microphone 510. Dynamic microphone 510 comprises a diaphragm 568, microphone body 588 and magneto coil element 586. In operation, sound waves cause movement of diaphragm 568 and attached magneto coil element 586. A magnet, for example magnet 598 or an additional magnet (not shown), produces a magnetic field which surrounds coil element 586. The motion of coil element 586 within this field causes an electrical signal representing the sound waves. These electrical signals, referred to herein as the microphone output, are then pre-processed by one or more functional components 596 positioned on printed circuit board (PCB) 594. Pre-processing of the microphone output may include amplification, filtering, digitization, etc.

As described above with reference to FIG. 3B, the pre-processed microphone signals are then wirelessly transmitted to main implantable component 242. More specifically, the pre-processed electrical signals are provided via electrical feed through 576 to microphone coil 574 where the electrical signals are inductively transferred to primary coil 260. Electrical feed through 576 is used because at least microphone 510 and the other electrical components are hermetically sealed by seal 562. In the illustrative arrangement of FIG. 5B, magnet 598, microphone 510 and the electrical components are hermetically sealed by seal 562. A tuning capacitor 578 may be provided to facilitate the transfer of the electrical signals.

As would be appreciated, the pre-processing and transferring of the microphone output to main implantable component 242 requires at least some power to be provided. This power is provided by a local rechargeable power source 599. Local power source 599 may comprise, for example, a miniature rechargeable battery, capacitor, etc. As explained in detail above with reference to FIG. 3B, local power source 599 is recharged by power received from main implantable component 242.

As shown in FIG. 5B, implantable microphone system 502 includes a cover 564 to protect microphone 510. Cover 564 may be formed from a suitable biocompatible material such as titanium. Cover 564 may also include an isolation layer formed from, for example, silicone. Diaphragm 568 may comprise a portion of cover 564, or may be circumferentially surrounded by cover 564. Furthermore, diaphragm 568 may be affixed in position using a suitable fixation arrangement or element 566. This may include the use of corrugations and the like to facilitate deflection of diaphragm 568.

The embodiments of FIG. 5B have been described herein with reference to a specific dynamic microphone. It would be appreciated that embodiments of the present invention are not limited to any particular type of microphone structure or technology. For example, embodiments of the present invention may utilize other types of dynamic microphones, such as ribbon microphones. In alternative embodiments, capacitor or condenser microphones, such as electret or MEMS microphones may be utilized. In further embodiments, crystal or piezoelectric microphones may be used, while other embodiments use liquid microphones. An example of a liquid microphone is a water microphone in which a conductor on water is expanding or shrinking in response to an input sound wave.

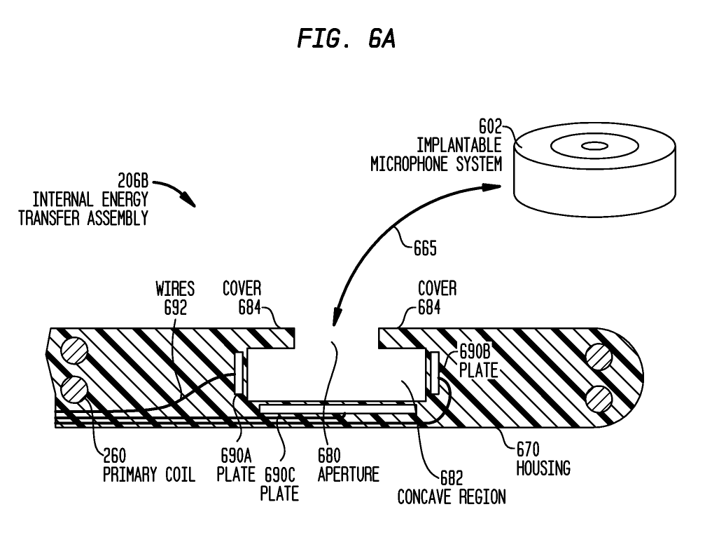

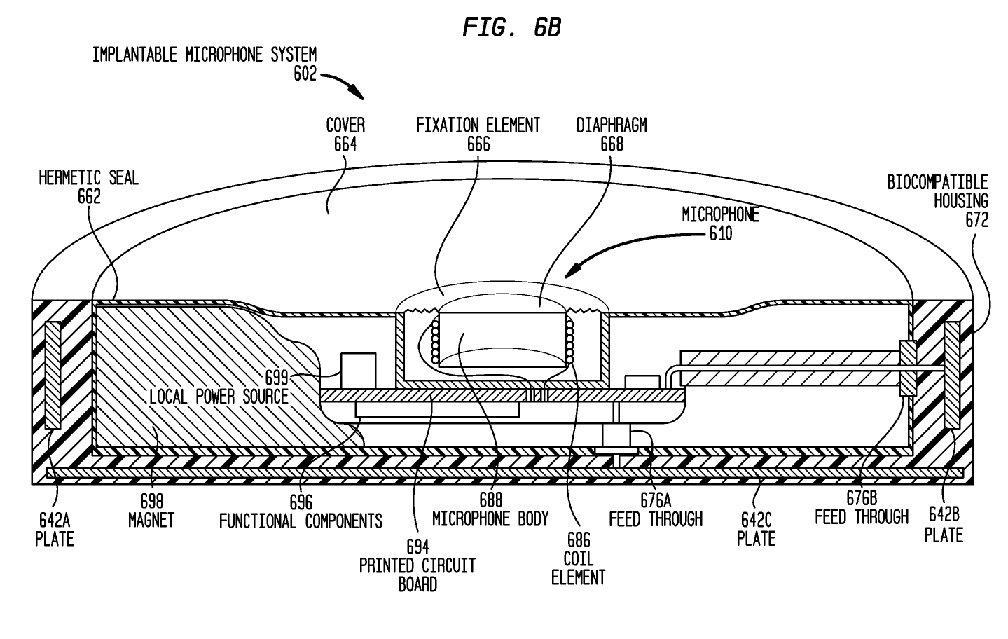

FIG. 6A is a cross-sectional view of internal energy transfer assembly 206, referred to as energy transfer assembly 206B, in accordance with embodiments of the present invention. As noted above, internal energy transfer assembly 206B includes a biocompatible housing 670 in which primary coil 260 is positioned. Wires (not shown) extending from primary coil 260 to components of main implantable component 242 may also be embedded in housing 570. Biocompatible housing 670 may comprise any suitable flexible material, such as silicone or other polymer.

As shown in FIG. 6A, housing 670 includes a concave region 682 in which an implantable microphone system 602 may be implanted. Concave region 682 is bound by a portion of biocompatible housing 670, shown as cover 684. Cover 684 has an aperture 680 therein through which implantable microphone system 602 is inserted. As noted housing 670 may comprise a flexible material, thus the orientation of cover 684 may be manipulated to enable insertion of implantable microphone system 602. Once positioned in housing 670, implantable microphone system 670 is substantially surrounded by housing, and cover 684 serves to retain in implantable microphone system 602 in the proper position. The positioning of implantable microphone system 602 into concave region 682 is shown by arrow 665. Details of implantable microphone system 602 are provided below with reference to FIG. 6B.

As described with reference to FIG. 6B, in this illustrative embodiment, implantable microphone system 602 is configured to capactively transfer pre-processed microphone signals to main implantable component 242. Because a wireless capacitive communication link is utilized, there is no direct, physical electrical connection between implantable microphone system 602 and the remaining components of internal energy transfer assembly 206B. A particular advantage of this implementation is that implantable microphone system 602 may be replaced or upgraded with only minimally invasive surgery. Specifically, as shown with reference to FIG. 7, because implantable microphone system 602 is located near the recipient's skin surface, only a small incision is required to access the system. This provides a surgeon with the ability to easily and safely access implantable microphone system 602, whether for repair, replacement or upgrade.

To enable the capacitive transfer capacitive with implantable microphone system 602, capacitive plates 690 are provided. Wires 692 extend from plates 690 through housing 670 to main implantable component 242.

It would be appreciated that an implanted microphone may be sensitive to both air-conducted sound as well as bone-conducted sound. However, typically in implanted microphones only the air-conducted sound is useful in evaluating a target sound signal, and the body or bone-conducted sounds typically comprises noise that degrades performance of the microphone. For example, body borne sound, such as breathing, may be conducted through the recipient's skull to an implanted microphone. As noted, housing 670 comprises a flexible material. In embodiments of the present invention, housing 670 provides passive vibration isolation for the implantable microphone system 602 and reduce the body conducted sound that is received thereby.

FIG. 6B is a cross-sectional view of implantable microphone system 602 of FIG. 6A. As would be appreciated, the illustrative arrangement of FIG. 6B is not to scale and has been enlarged for ease of illustration.

As shown, implantable microphone system 602 comprises a biocompatible housing 672 which may comprise, for example a flexible material such as silicon or other polymer. Positioned in biocompatible housing are capacitive plates 642. As described below, plates 642 cooperate with plates 690 shown in FIG. 6A to transfer power and pre-processed microphone signals between implantable microphone system 602 and main implantable component 242.

Implantable microphone system 602 further comprises a magnet 698. As explained above with reference to FIG. 3A, magnet 698 is configured to align an external device with internal energy transfer assembly 206. This alignment enables efficient transfer of power and/or data between the external device and internal energy transfer assembly 206. Disposed in magnet 698 is microphone 610. That is, microphone 610 is at least partially surrounded by magnet 698. For example, in certain embodiments, magnet 698 is positioned around and below microphone 610. In other embodiments, magnet 698 is positioned around microphone 610. In a specific such embodiment, microphone 610 has a circumference which is substantially surrounded by magnet 698. In certain embodiments, at least some spacing between magnet 698 and microphone 610 is provided to minimize electrical losses caused by incidental Eddy currents inside the magnet.

In the illustrative embodiments of FIG. 6B, microphone 610 comprises a magneto-dynamic, or simply dynamic, microphone 610. Dynamic microphone 610 comprises a diaphragm 668, microphone body 688 and magneto coil element 686. In operation, sound waves cause movement of diaphragm 668 and attached coil element 686. A magnet, for example magnet 698 or an additional magnet (not shown), produces a magnetic field which surrounds coil element 686. The motion of coil element 686 within this field causes an electrical signal representing the sound waves. These electrical signals, referred to herein as the microphone output, are then pre-processed by one or more functional components 696 positioned on printed circuit board (PCB) 694. Pre-processing of the microphone output may include amplification, filtering, digitization, etc.

As described above with reference to FIG. 3B, the pre-processed microphone signals are then wirelessly transmitted to main implantable component 242. More specifically, the pre-processed electrical signals are provided via electrical feed throughs 676 to plates 642A, 642B, and 642C. The pre-processed microphone signals are then capactively transferred by plates 674 to corresponding opposing plates 690 in internal energy transfer assembly 206B outside implantable microphone system 602. Electrical feed throughs 676 are used because at least microphone 610 and the other electrical components are hermetically sealed by seal 662. In the illustrative arrangement of FIG. 6B, magnet 698, microphone 610 and the electrical components are hermetically sealed by seal 662.

As would be appreciated, the pre-processing and transferring of the microphone output to main implantable component 242 requires at least some power to be provided. This power is provided by a local rechargeable power source 699. Local power source 699 may comprise, for example, a miniature rechargeable battery, capacitor, etc. As explained in detail above with reference to FIG. 3B, local power source 699 is recharged by power received from main implantable component 242.

As shown in FIG. 6B, implantable microphone system 602 includes a cover 664 to protect microphone 610. Cover 664 may be formed from a suitable biocompatible material such as titanium. Cover 664 may also include an isolation layer formed from, for example, silicone. Diaphragm 668 may comprise a portion of cover 664, or may be circumferentially surrounded by cover 664. Furthermore, diaphragm 668 may be affixed in position using a suitable fixation arrangement or element 666. This may include the use of corrugations and the like to facilitate deflection of diaphragm 668.

The embodiments of FIG. 6B have been described herein with reference to a specific dynamic microphone. It would be appreciated that embodiments of the present invention are not limited to any particular type of microphone structure or technology. For example, embodiments of the present invention may utilize other types of dynamic microphones, such as ribbon microphones. In alternative embodiments, capacitor or condenser microphones, such as electret or MEMS microphones may be utilized. In further embodiments, crystal or piezoelectric microphones may be used, while other embodiments use liquid microphones. An example of a liquid microphone is a water microphone in which a conductor on water is expanding or shrinking in response to an input sound wave.

As noted, FIGS. 6A and 6B use an inductive link to receive power from an external device, and a capacitive arrangement to transfer power/data between implantable microphone assembly 602 and main implantable component 242. This arrangements has an advantage over embodiments which use the primary coil for subcutaneous and transcutaneous communication in that subcutaneous and transcutaneous may occur concurrently or simultaneously in the embodiments of FIGS. 6A and 6B. This avoids the need to cease operation of the implanted microphone during charging operations. The ability to provide simultaneous subcutaneous and transcutaneous is enabled by the fact that two different non-overlapping communication links are used for each type of transfer.

FIG. 7 is a schematic diagram of illustrating an exemplary implanted location of components of cochlear implant 200 in accordance with embodiments of the present invention. As detailed above, implantable microphone system 202 and main implantable component 242 are enclosed in biocompatible housing 470. Postiioned in internal energy transfer assembly 206 is an implantable microphone system 202. Implant housing 470 is manufactured from one or more biocompatible materials including but not limited to metals and their alloys; polymers and polymer composites; and/or ceramics and carbon-based materials. Utilization of other materials that satisfy the requirements of being biologically acceptable to the host tissue and remaining stable and functional are also contemplated, and are considered to be within the scope of the present invention.

In the exemplary embodiment shown in FIG. 7, the illustrated components of cochlear implant 200 are embedded in the recipient's skin/tissue 250 so that a base wall housing 270 is proximate to a bone or other rigid body structure such as, for example mastoid bone 782. Sound waves 103 pass through tissue 232 and strike diaphragm 568 of implantable microphone system 202. As discussed in detail above, the vibration of diaphragm 568 is used to generate electrical signals used as a sound input for totally implantable cochlear implant 200.

As is well know, most conventional cochlear implants use microphones positioned externally to the recipient. The use of an implantable microphone raises practical difficulties that are addressed by embodiments of the present invention. For example, an issue that arises with implantable microphones is the transfer of an externally generated sound signal to an internally positioned diaphragm.

In operation, a sound signal 103 impinges upon skin/tissue 250 and is relayed to diaphragm 568. In embodiments of the present invention implantable microphone system 202 is positioned close to the skin surface to minimize the loss of sound through skin/tissue 250. In further embodiments, during implantation, the surgeon ensures that skin/tissue 250 abuts diaphragm 568 to increase the transfer efficiency of sound signals between the tissue and the diaphragm. However, regardless of whether skin/tissue is in physical contact with diaphragm 568, it would be appreciated that the body fluid would substantially fill any gaps between the tissue and the diaphragm thereby increasing the transfer efficient of sound from the tissue to the diaphragm.

As noted above, in embodiments of the present invention, implantable microphone system 202 is preferably removable from internal energy transfer assembly 206. However, in certain embodiments it may be beneficial to increase the transfer efficiency of sound signals from the skin/tissue 250 to diaphragm 568 by securing the diaphragm to the tissue. In certain embodiments, this may be done through an appropriate choice of material for diaphragm 568, texturing the diaphragm, or providing mechanical interlocks between the tissue and the diaphragm. Exemplary mechanical interlocks may comprise, for example, eyelets or other structural feature on diaphragm 568 which would encourage fibruous tissue growth therewith. Such features may be microscopic in size and would be designed to minimize the areas where bacteria could potentially gather or grow. As would be appreciated, the securing of diaphragm 568 to skin/tissue 250 is not desirable in all circumstances, but may provide a surgeon with the above noted advantages, if desired.

It would be appreciated that in certain embodiments of the present invention, diaphragm 568 could be treated with a pharmaceutical agent prior to, during, or after implantation of cochlear implant 200. The pharmaceutical agent may comprise, for example, an antibacterial coating to reduce the chance of infection.

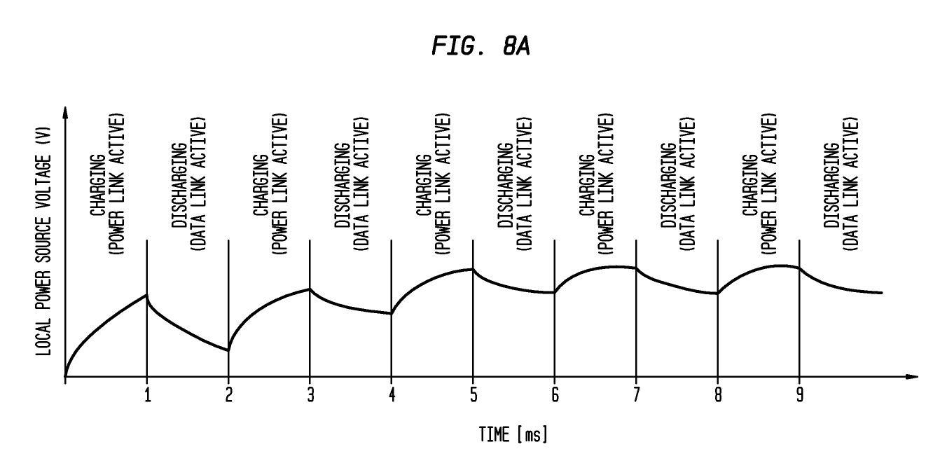

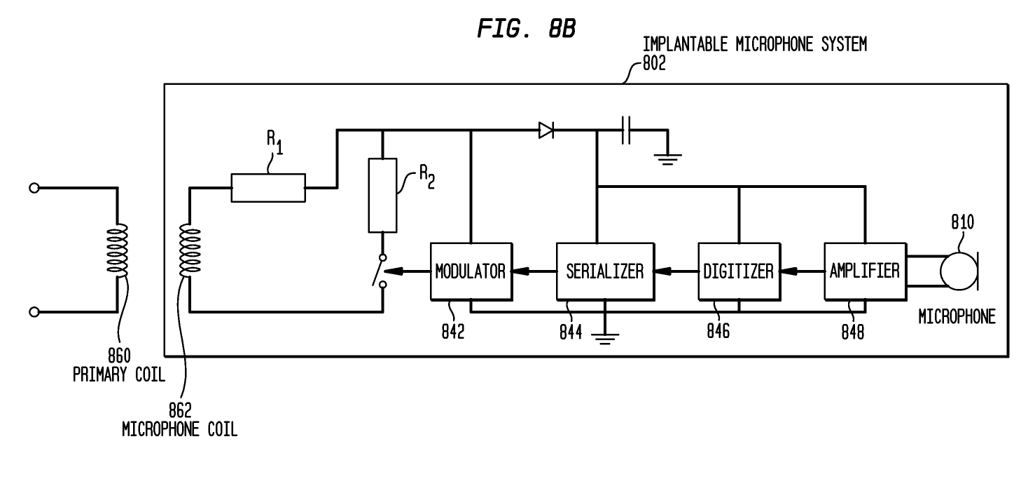

As noted, cochlear implant 200 is configured to recharge a local power source in implantable microphone system 202 using a power communication link, simply power link herein, between main implant component 242 and implantable microphone system 202. Furthermore, implantable microphone system 202 is configured to provide pre-processed microphone signals to main implantable component 242 via a data communication link, simply data link herein. In certain embodiments, the transfer of power between implantable microphone system 202 and main implantable component 242 occurs via a discontinuous power link interleaved with a data link transmitting pre-processed microphone signals from the microphone system to the main implantable component. FIG. 8A is a diagram illustrating such embodiments. Specifically, FIG. 8A illustrates the voltage of a local power source over successive time intervals during the discontinuous transfer of power.

As shown in FIG. 8A, the discontinuous power link has a duty cycle of fifty-percent (50%). In other words, half of the successive time intervals are allocated to the power link, while the other half of the successive time intervals are allocated to the data link. A discontinuous power link with a duty cycle of 50% will charge a local power source to the voltage shown in FIG. 8A. As shown, during the time intervals allocated to the data link, the local power source will be slightly discharged due to the processing and transmission that occurs. However, the overall voltage of the local power source remains at a suitable level to fully power the implantable microphone assembly. Buffering and compression techniques are used to fit in the pre-processed microphone signals into the time intervals allocated to the data link.

As noted above with reference to FIG. 3B, various transfer schemes may be implemented to transfer power and pre-processed microphone signals between implantable microphone system 202 and main implantable component 242. FIG. 8B is a schematic view of one embodiment of an implantable microphone system 802 illustrating one exemplary arrangement for subcutaneous transfer of power and data between the microphone system and a primary coil 860. In the embodiments of FIG. 8B, load modulation is used to transfer the power and pre-processed microphone signals. As shown, implantable microphone system 202 comprises a microphone 810 to receive a sound signal, and a plurality of functional components which pre-process the microphone output, and which facilitate the load modulation transfer. The functional components include an amplifier 848, digitizer 846, serializer 844, a modulator 842, and a microphone coil 862.

Using the arrangement illustrated in FIG. 8B, the power and data transfer between implantable microphone system 802 and primary coil 860 may occur substantially simultaneously. In the illustrative embodiments of FIG. 8B, the average load represented by one or more of the functional components is held constant. However, the electrical signal representing the microphone output will modulate the load. Modulator 842 then modulates the signal amplitude using a switch (On-Off-Keying). It would be appreciated that other variations on load modulation and/or amplitude, frequency or phase modulation may be used to transfer the power and data, and that the embodiments of FIG. 8B are provided for illustration only. Furthermore, as noted with reference to FIG. 3B, embodiments of the present invention are not limited to any specific data/power transfer scheme.

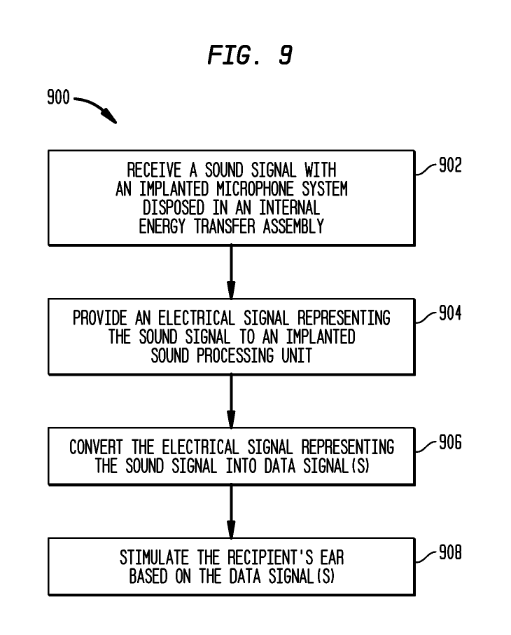

FIG. 9 is a flowchart illustrating a method 900 performed by a hearing prosthesis in accordance with embodiments of the present invention. As shown, method 900 begins at block 902 where the hearing prosthesis receives a sound signal with an implanted microphone system disposed or positioned in an internal energy transfer assembly. At block 904, an electrical signal presenting the sound signal is provided to an implanted sound processing unit. At block 906, the sound processing unit converts the electrical signal representing the sound signal into data signals, and at block 908 the hearing prosthesis stimulates the recipient's ear using the generated data signals. It would be appreciated that the additional of steps to or the modification of the above steps of method 900 are within the scope of the present invention.

As noted above, embodiments of the present invention may be implemented in any partially or fully implantable hearing prosthesis now known or later developed. For example, embodiments may implemented in acoustic hearing aids, auditory brain stimulators, middle ear mechanical stimulators, hybrid electro-acoustic prosthesis or other prosthesis that electrically, acoustically and/or mechanically stimulate components of the recipient's outer, middle or inner ear.

Furthermore, embodiments of the present invention have been discussed primarily with reference to an implantable microphone system disposed in an internal energy transfer assembly. However, it would be appreciated that the implantable microphone system may be positioned in any implanted component in which it is desirable to align the implanted component with an external device.

All documents, patents, journal articles and other materials cited in the present application are hereby incorporated by reference.

The invention described and claimed herein is not to be limited in scope by the specific preferred embodiments herein disclosed, since these embodiments are intended as illustrations, and not limitations, of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

1. A cochlear implant totally implantable in a recipient comprising:
an internal energy transfer assembly configured to receive power from an external device and having an implantable microphone system removably positioned therein configured to receive a sound signal and to generate electrical signals representing the received sound signal;
a main implantable component having a sound processing unit configured to convert the electrical signals into data signals; and
an electrode assembly implantable in the recipient's cochlea configured to deliver to the cochlea electrical stimulation signals generated based on the data signals.

2. The cochlear implant of claim 1, wherein the implantable microphone system comprises:
a magnet; and
a microphone disposed in the magnet configured to receive the sound signal and generate an electrical microphone output representing the sound signal.

3. The cochlear implant of claim 2, wherein the implantable microphone system further comprises:
one or more components configured to convert the microphone output into a pre-processed microphone output.

4. The cochlear implant of claim 2, wherein the implantable microphone system further comprises:
a local rechargeable power source.

5. The cochlear implant of claim 2, wherein the implantable microphone system is configured to wirelessly transmit an electrical representation of the microphone output to the main implantable component.

6. The cochlear implant of claim 4, wherein main implantable component comprises a rechargeable power source, and wherein the implantable microphone system is configured to wirelessly receive power from the main implantable component to recharge the local power source.

7. The cochlear implant of claim 1, wherein the internal energy transfer assembly further comprises:
a primary receiving coil configured to receive at least one of power and data from an external device.

8. The cochlear implant of claim 7, wherein the implantable microphone system comprises a microphone coil configured to inductively transmit the electrical signals representing the sound signal to the primary coil, and wherein the primary coil provides the electrical stimulation signals to the main implantable component.

9. The cochlear implant of claim 7, wherein the implantable microphone system comprises one or more capacitive plates, and wherein the internal energy transfer assembly further comprises one or more capacitive plates capacitively coupled to the plates in the microphone system, and wherein the microphone system is configured to use the capacitive coupling to provide the electrical signals representing the sound signal to the main implantable component.

10. The cochlear implant of claim 1, wherein the internal energy transfer assembly further comprises:
at least one capacitive plate configured to receive at least one of power and data from an external device.

11. The cochlear implant of claim 10, wherein the implantable microphone system comprises one or more capacitive plates configured to be capacitively coupled to the at least one capacitive plate, and wherein the microphone system is configured to use the capacitive coupling to provide the electrical signals representing the sound signal to the main implantable component.

12. The cochlear implant of claim 10, wherein the internal energy transfer assembly comprises a transmitting/receiving coil, and wherein the implantable microphone system comprises a microphone coil configured to transmit the electrical signals representing the sound signal to the transmitting/receiving coil, and wherein the transmitting/receiving coil provides the electrical stimulation signals to the main implantable component.

13. The cochlear implant of claim 1, wherein the implant is configured to at least one of receive data from, and transmit data to, the external device via the internal energy transfer assembly.

14. A hearing prosthesis at least partially implantable in a recipient comprising:
an implantable internal energy transfer assembly configured to receive power from an external device, and having an implantable microphone system removably positioned therein configured to receive a sound signal and to generate electrical signals representing the received sound signal;
a main implantable component having a sound processing unit configured to convert the electrical signals into data signals; and
an output stimulator configured to stimulate the recipient's ear based on the data signals.

15. The hearing prosthesis of claim 14, wherein the implantable microphone system comprises:
a magnet; and
a microphone disposed in the magnet configured to receive the sound signal and generate an electrical microphone output representing the sound signal.

16. The hearing prosthesis of claim 14, wherein the implantable microphone system further comprises:
one or more components configured to convert the microphone output into a pre-processed microphone output.

17. The hearing prosthesis of claim 14, wherein the implantable microphone system further comprises:
a rechargeable local power source.

18. The hearing prosthesis of claim 15, wherein the implantable microphone system is configured to wirelessly transmit an electrical signal representing the microphone output to the main implantable component.

19. The hearing prosthesis of claim 17, wherein main implantable component comprises a rechargeable power source, and wherein the implantable microphone system is configured to wirelessly receive power from the main implantable component to recharge the local power source.

20. The hearing prosthesis of claim 14, wherein the internal energy transfer assembly further comprises:
a primary receiving coil configured to receive at least one of power and data from an external device.

21. The hearing prosthesis of claim 20, wherein the implantable microphone system comprises a microphone coil configured to inductively transmit the electrical signals representing the sound signal to the primary coil, and wherein the primary coil provides the electrical stimulation signals to the main implantable component.

22. The hearing prosthesis of claim 20, wherein the implantable microphone system comprises one or more capacitive plates, and wherein the internal energy transfer assembly further comprises one or more capacitive plates capacitively coupled to the plates in the microphone system, and wherein the microphone system is configured to use the capacitive coupling to provide the electrical signals representing the sound signal to the main implantable component.

23. The hearing prosthesis of claim 14, wherein the internal energy transfer assembly further comprises:
at least one capacitive plate configured to receive at least one of power and data from an external device.

24. The hearing prosthesis of claim 23, wherein the implantable microphone system comprises one or more capacitive plates configured to be capacitively coupled to the at least one capacitive plate, and wherein the microphone system is configured to use the capacitive coupling to provide the electrical signals representing the sound signal to the main implantable component.

25. The hearing prosthesis of claim 23, wherein the internal energy transfer assembly comprises a transmitting/receiving coil, and wherein the implantable microphone system comprises a microphone coil configured to transmit the electrical signals representing the sound signal to the transmitting/receiving coil, and wherein the transmitting/receiving coil provides the electrical stimulation signals to the main implantable component.

26. The hearing prosthesis of claim 14, wherein the prosthesis is configured to at least one of receive data from, and transmit data to, the external device via the internal energy transfer assembly.

27. The hearing prosthesis of claim 14, wherein the hearing prosthesis is a cochlear implant.

28. The hearing prosthesis of claim 14, wherein the hearing prosthesis is a middle ear mechanical stimulator.

29. The hearing prosthesis of claim 14, wherein the hearing prosthesis is an electro-acoustic stimulator.

30. The hearing prosthesis of claim 16, wherein the hearing prosthesis is an acoustic hearing aid.

31. A method for evoking a hearing percept in a recipient comprising:
receiving a sound signal via an implantable microphone system removably positioned in an implantable internal energy transfer assembly configured to receive power from an external device;
providing an electrical signal representing the sound signal to a main implantable component having a sound processing unit;
converting, with the sound processing unit, the electrical signal representing the sound signal into one or more data signals; and
stimulating the recipient's ear based on the one or more data signals.

32. The method of claim 31, wherein the implantable microphone system comprises a magnet, a microphone disposed in the magnet, and a one or more functional components, and wherein receiving the sound signal comprises:
converting, with the microphone, the received sound to an electrical microphone output representing the sound signal; and
pre-processing the microphone output with the one or more functional components.

33. The method of claim 31, wherein providing the electrical signal representing the sound signal to the main implantable component comprises:
wirelessly transmitting a representation of the electrical signal to the main implantable component.

34. The method of claim 31, wherein the implantable microphone system comprises a local rechargeable power source, and wherein the main implantable component comprises a rechargeable power source, and wherein the method further comprises:
wirelessly transmitting power from the main implantable component to the implantable microphone system to recharge the local power source.
